(19) United States
(12) Patent Application Publication
(10) Pub. No.: US 2020/0101492 A1
(43) Pub. Date: Apr. 2, 2020

(54) CAPACITIVE TRANSDUCER, MANUFACTURING METHOD THEREOF, AND IMAGE FORMING APPARATUS

(71) Applicant: CANON KABUSHIKI KAISHA, Tokyo (JP)

(72) Inventors: Takahiro Akiyama, Atsugi-shi (JP); Takayuki Sumida, Kawasaki-shi (JP)

(21) Appl. No.: 16/581,146

(22) Filed: Sep. 24, 2019

(30) Foreign Application Priority Data
Sep. 28, 2018 (JP) 2018-185433

(51) Int. Cl. B06B 1/02; A61B 8/08; A61B 8/00

(52) U.S. Cl. CPC B06B 1/0292; B06B 1/0215; B06B 2201/76; A61B 8/54; A61B 8/4483; A61B 8/52

(57) ABSTRACT

A capacitive transducer includes at least one element. The element is provided on a flexible substrate. The elements can be deformed and placed in a plurality of directions on the basis of the flexibility of the substrate.

### BACKGROUND

### Field

One disclosed aspect of the embodiments relates to a capacitive transducer, a manufacturing method thereof, and an image forming apparatus.

### Description of the Related Art

Capacitive transducers fabricated with micromachining techniques are under development to be substituted for piezoelectric elements. Hereinafter, a capacitive transducer may occasionally be referred to as a capacitive micro-machined ultrasound transducer (CMUT).

With use of such a CMUT, ultrasonic waves can be transmitted and received by using vibrations of a membrane. Further, since the CMUT can produce shorter ultrasonic pulses due to excellent broadband characteristics of the CMUT, spatial resolution in an ultrasonic wave transmission direction can be improved. Moreover, an evaluation with fewer ultrasound probes become possible even in a case where an evaluation with a plurality of ultrasound probes is conventionally required. Thus, the CMUT leads to simplification of the device and a reduction in cost. Further, the broadband characteristics are useful in a harmonic imaging technique.

Examples of general ultrasound probes include a convex ultrasound probe. A piezoelectric element is mounted on a curved surface to allow a wide area of tomographic images mainly of, for example, the abdomen. Due to a usage of a silicon substrate, it is difficult to mount, on a curved surface, a capacitive transducer fabricated mainly with a semiconductor manufacturing process technique. A method has been proposed that splits a fabricated capacitive transducer into small parts and places the small parts along guides previously formed on a desired curved mount surface (Japanese Patent Application Laid-Open No. 2017-148258).

The CMUT has an acoustic impedance close to that of a medium as compared to a known transducer provided with a piezoelectric element. Thus, the CMUT has a wider frequency band and a short transmission pulse of an ultrasonic wave, and a high-resolution image can be obtained accordingly. However, since the CMUT is fabricated on a semiconductor substrate, it is difficult to mount the CMUT on a curved surface due to the rigidity of the substrate. An ultrasound probe acquires in vivo information. In order to obtain the in vivo information, the ultrasound probe needs to come into contact as close as possible with an object to be measured, via gel or liquid for matching acoustic impedance. Therefore, if an ultrasound probe having the CMUT on a free curved surface is achieved, in vivo information can be extracted by an ultrasonic signal even from a region of a complicated shape. Moreover, an artifact occurring as a result of diffraction due to the distance between elements influences the quality of an ultrasound image.

### SUMMARY

In order to minimize the influence of the diffraction artifact and the probe contact issues as above, the distance between elements needs to be reduced as much as possible, and it is required to mount elements densely. According to an aspect of the embodiments, a capacitive transducer is a capacitive transducer including at least one element. The element includes at least one cell. The cell includes a first electrode, a first insulating film on the first electrode, and a vibrating membrane having a second insulating film and a second electrode, which face the first insulating film across a cavity. The element is provided on a flexible substrate. The substrate includes, on a surface thereof, a surface electrode and wiring electrically connected to the surface electrode. The first and second electrodes are electrically connected to the surface electrode. The elements can be deformed and placed in a plurality of directions on the basis of the flexibility of the substrate.

Further features of the disclosure will become apparent from the following description of exemplary embodiments with reference to the attached drawings.

### BRIEF DESCRIPTION OF THE DRAWINGS

FIGS. 1A to 1C are a top view, a cross-sectional view taken along line A-A′, and a cross-sectional view taken along line B-B′, respectively, for describing a capacitive micro-machined ultrasound transducer (CMUT) according to an exemplary embodiment.

FIG. 2 is a cross-sectional view for describing a CMUT according to an exemplary embodiment.

FIG. 3 is a cross-sectional view for describing a CMUT according to an exemplary embodiment.

FIGS. 4A and 4B are a top view and a cross-sectional view taken along line C-C′, for describing a CMUT according to an exemplary embodiment.

FIG. 5 is a diagram for describing an example of a CMUT according to a third exemplary embodiment.

FIG. 6 is a diagram for describing an example of a CMUT according to a fourth exemplary embodiment.

FIG. 7 is a diagram for describing an example of a CMUT according to a fifth exemplary embodiment.

FIGS. 8A to 8H are diagrams for describing a method of manufacturing a CMUT according to an exemplary embodiment.

FIGS. 9A to 9C are diagrams for describing the method of manufacturing a CMUT according to the exemplary embodiment.

FIG. 10 is a diagram for describing an example of an image forming apparatus using a CMUT according to a seventh exemplary embodiment.

### DESCRIPTION OF THE EMBODIMENTS

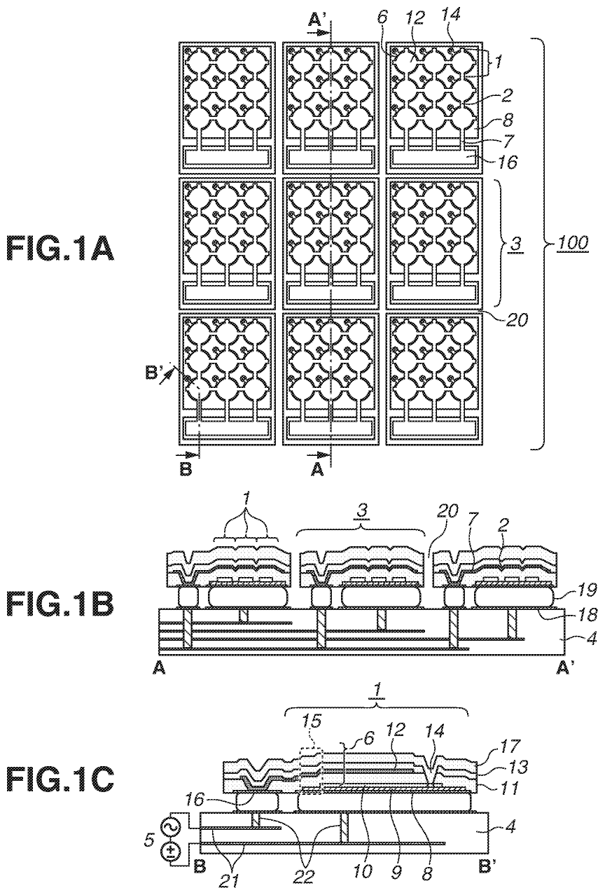

An exemplary embodiment of the disclosure is described hereinafter with reference to FIGS. 1A, 1B, and 1C. FIG. 1A is a top view of a capacitive micro-machined ultrasound transducer (CMUT) according to the present exemplary embodiment. FIGS. 1B and 1C are cross-sectional views taken along line A-A′ and line B-B′ of FIG. 1A.

A CMUT 100 according to the present exemplary embodiment includes elements 3 each configured to include a plurality of cells 1. Cells 1 in one element 3 are electrically connected by wiring 2. In FIG. 1A, although the number of the cells 1 included in one element 3 is nine, any number is acceptable. The arrangement shape of the cells 1 has an equally spaced tetragonal lattice structure. However, the arrangement shape may have a hexagonal close-packed structure or an unequally spaced structure. In FIG. 1A, a vibrating membrane 6 configuring the cell 1 may have a shape of a circle, an ellipse, a polygon such as a hexagon, or a rectangle.

The cell 1 is configured to include a first electrode 8, a first insulating film 9, and the vibrating membrane 6 facing the first insulating film 9 across a cavity 10. The vibrating membrane 6 includes a second insulating film 11, a third insulating film 13, and a second electrode 12 between the second insulating film 11 and the third insulating film 13, and a sealing film 17. The vibrating membrane 6 is supported by a supporting portion 15 in a vibrating manner The sealing film 17 may be removed except the periphery of a sealing portion 14. In this case, constructional elements of the vibrating membrane 6 include the second insulating film 11, the second electrode 12, and the third insulating film 13. The cell 1 or the element 3 is bonded to a flexible (pliable) substrate 4 via an adhesive substance 19. Owing to the flexibility of the substrate 4, the elements 3 can be deformed and placed in a plurality of directions.

In the CMUT 100 illustrated in FIG. 1A, although the number of the elements 3 is nine, the number may be one or more, and any number can be used. If there is a plurality of the elements 3, one of the first electrode 8 and the second electrode 12 is electrically insulated. Further, either of the first electrode 8 and the second electrode 12 may be common to the plurality of the elements 3. In other words, each of the first electrodes 8 of the plurality of the elements 3, or each of the second electrodes 12 of the plurality of the elements 3, may be electrically connected together.

The first electrode 8 or the second electrode 12 is used as an electrode that applies a direct current (DC) bias voltage, or an electrode for applying or outputting an electrical signal. The electrode that applies a DC bias voltage is common to the elements 3. The configuration where a DC bias voltage is common to the elements 3 is acceptable. However, the electrodes that transmit and receive signals are electrically separated on an element-by-element basis.

The material of the cell 1 is described. A desired material of the first electrode 8 is metal. A metal such as tungsten, molybdenum, titanium, aluminum, neodymium, chromium, or cobalt can be used. A laminated structure where a plurality of layers of these metals is laminated, a compound of these metals, an alloy of these metals, a compound of these metals and silicon and copper, or an alloy of these metals and silicon and copper is also acceptable. A semiconductor or compound semiconductor where these metals include a high concentration of impurities is also acceptable.

The cavity 10 is covered by the first insulating film 9, the second insulating film 11, and the sealing portion 14. The cavity 10 is sealed under sufficiently reduced pressure as compared to atmospheric pressure. The sealing of the cavity 10 prevents liquid from entering the cavity 10 during a process after sealing or during use. Moreover, the reduced pressure increases the sensitivity of the CMUT. The first electrode 8 and the second electrode 12 are insulated by insulators including the cavity 10. In the case of FIG. 1B, the first electrode 8 and the second electrode 12 are insulated by the first insulating film 9, the second insulating film 11, and the third insulating film 13. These insulating films 9, 11, and 13 include silicon oxide, and silicon nitride. A desired material of the second electrode 12 is metal. For example, tungsten, molybdenum, titanium, aluminum, neodymium, chromium, cobalt, a compound or alloy thereof, or a compound or alloy of silicon and copper is acceptable.

The principle of operation of the CMUT 100 of the present exemplary embodiment is described. If ultrasonic waves are transmitted and received by the CMUT 100, a voltage application unit 5 produces a potential difference between the first electrode 8 and the second electrode 12 through wiring 21 and a via 22, which are installed in the substrate 4. When an AC voltage, in addition to a DC voltage, is applied to between the first electrode 8 and the second electrode 12, the vibrating membrane 6 vibrates due to a temporal change of electrostatic force. The vibrations of the vibrating membrane 6 range from several tens of kilohertz to several tens of megahertz, which is the frequency band of ultrasound. The principle is to produce ultrasonic waves by directly vibrating a substance on the vibrating membrane 6. In this manner, the CMUT 100 according to the present exemplary embodiment converts an electrical signal into vibrations of the vibrating membrane 6, and the vibrations produces ultrasonic waves. Accordingly, the CMUT 100 can transmit the ultrasonic waves.

On the other hand, when the CMUT 100 receives ultrasonic waves, the vibrating membrane 6 including the second electrode 12 vibrates, and thus the capacitance of the element 3 is changed. With the change of the capacitance, an alternating current flows through an output signal electrode. In this manner, the conversion of ultrasonic waves into an electrical signal allows the CMUT 100 to receive the ultrasonic waves. In a case of a capacitive transducer, when ultrasonic waves are received, the change of the capacitance produces a very small alternating current, which is output. The current is turned into a voltage signal amplified by a circuit such as an operation amplifier. The voltage signal is discretized by an analog-to-digital (A/D) converter and then processed.

The structure of the CMUT 100 of the present exemplary embodiment is described. Each element 3 of the CMUT 100 is bonded onto the flexible substrate 4. The elements 3 are structurally separated from each other. The width of a groove 20 between the elements 3 is set to be greater than the height of the element 3. Thus, the CMUT 100 can be flexibly deformed, and can touch and come into close contact with curved surfaces of various forms. The height of the element 3 is approximately several micrometers, and the width of the groove 20 is substantially equal to the height of the element 3. Thus, it is possible to have an array of the elements 3 of a densest pitch. Consequently, artifacts caused by the diffraction of ultrasonic waves can be reduced. Hence, an excellent ultrasound image can be provided. Further, the height of the element 3 from a top surface to a bottom surface can be set to be three micrometers or less, and thus the distance between adjacent elements 3 can be set to be twice or less the height of the element 3 from the top surface to the bottom surface of the element 3. Here, the height of the element 3 is the distance from a surface on the substrate side (e.g., the bottom surface) to a surface opposite to the substrate side (e.g., the top surface) of the element 3.

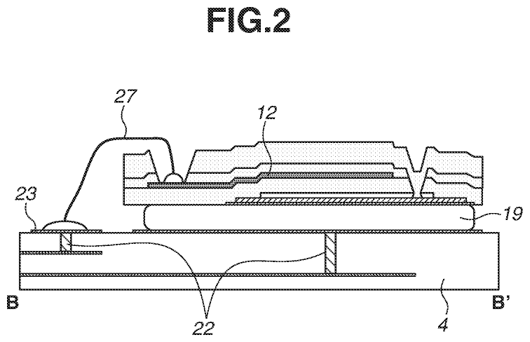

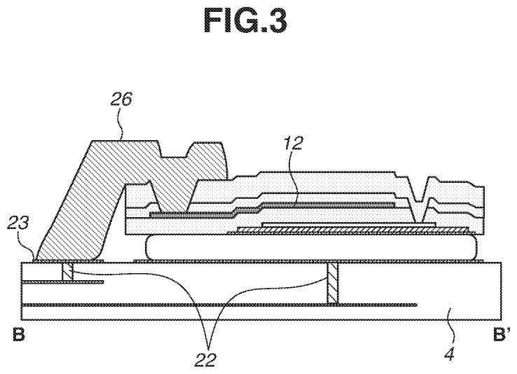

The second electrode 12 is extracted along a lead wire 7. The second electrode 12 is simply required to have a structure where an electrical signal can be extracted to reach an electrode 23 installed on the substrate 4 while being insulated and separated from the first electrode 8. The extraction structure may be point-to-point construction 27 (as illustrated in FIG. 2) such as wire bonding, pattern wiring 26 (as illustrated in FIG. 3) using screen printing or photolithography, or drawing wiring by an inkjet printer or other structures. In FIG. 1C, 2, or 3, a structure that extracts the second electrode 12 is illustrated. However, the first electrode 8 may be extracted in a similar structure, or both the first electrode 8 and the second electrode 12 may be extracted in a similar method. Furthermore, the second electrode 12 may be extracted to reach an electrode 16 on the bottom surface of the element 3, which is insulated and separated from the first electrode 8, as illustrated in FIG. 1C. Thus, the second electrode 12 may be electrically connected to the substrate 4 side. Consequently, the elements 3 can be installed more densely than those illustrated in FIGS. 2 and 3. The electrodes 8 and 16 and the substrate 4 are electrically and structurally connected to surface electrodes 18 formed on the substrate 4 in a conductive adhesion layer 19. The adhesion layer 19 may be a conductive resin or an alloy such as solder. The substrate 4 includes the wiring layer 21 and the via 22. An electrical signal traveling through the wiring layer 21 and the via 22 is finally transferred to an image forming apparatus.

As illustrated in FIG. 1C, the electrode 16 and the first electrode 8 on the bottom surface of the element 3 are separated by the second insulating film 11. The electrode separation structure can also be achieved using the first insulating film 9. The height of the element 3 is several micrometers, and the width thereof is several hundred micrometers. Since the structure is very thin and flat, it is desirable that the element 3 has a high rigidity. Silicon oxide and silicon nitride are sufficient as the material of the electrode separation structure. However, silicon nitride having a higher Young's modulus is preferred.

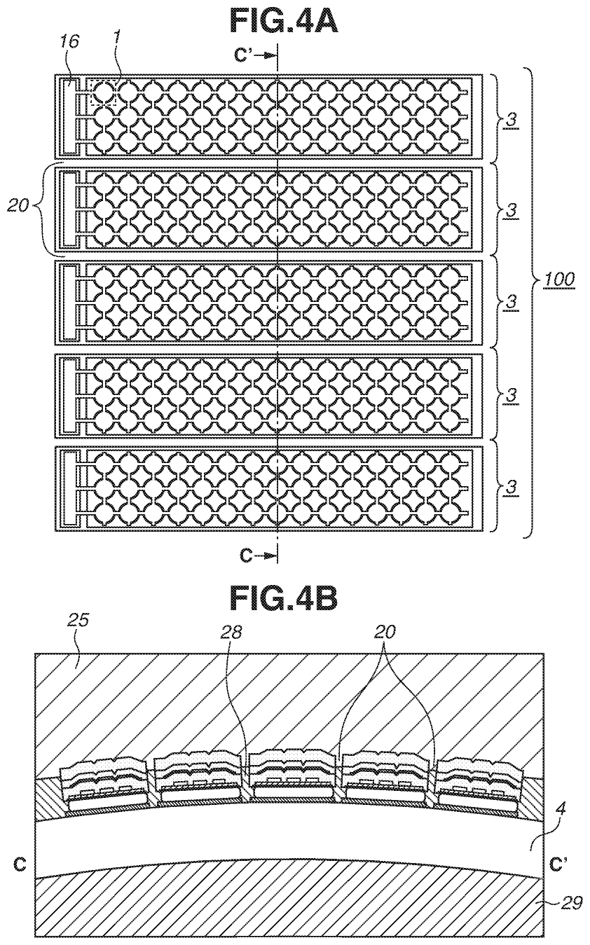

Moreover, as illustrated in FIG. 4A, the elements 3 may be arranged one-dimensionally. The grooves 20 are formed between the elements 3 to structurally separate the elements 3. As illustrated in FIG. 4B, upper parts of the elements 3 may be covered with a resin material 25. The resin material 25 has a role to protect the elements 3 and the surroundings thereof. The resin material 25 is molded to serve as an acoustic lens. The grooves 20 and gaps in the adhesion layer 19 may be buried with a resin 28 having insulation properties. Consequently, it is possible to prevent foreign substances from being introduced into the grooves 20, and to increase insulation reliability and structural reliability.

In FIGS. 4A and 4B, the flexible substrate 4 to which the elements 3 have been bonded is bonded to a base 29 having a curved surface. Consequently, the CMUT 100 of the present exemplary embodiment can provide a convex shaped linear probe. The base 29 may have a concave surface. In this case, a concave shaped linear probe can also be achieved. The base 29 may have a curved surface in the direction of the long side of the element 3. Further, the curvature of the curved surface of the base 29 is not necessarily uniform.

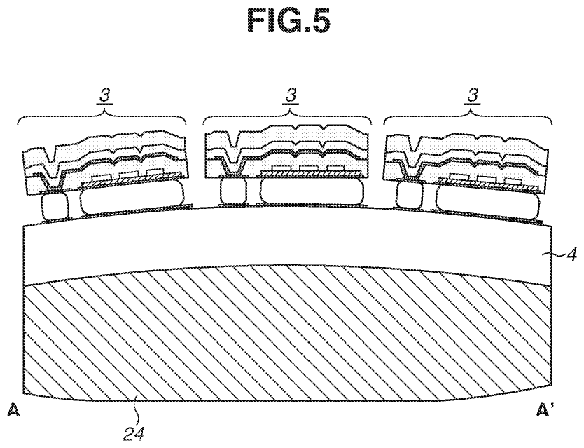

Ultrasonic waves produced or received by the element 3 also propagate and reflect in the direction of the substrate 4, and thus may influence a received signal or image quality. Therefore, it is desirable that the substrate 4 have acoustic attenuation properties. Further, as illustrated in FIG. 5, an acoustic attenuation member 24 may be installed on the back surface of the substrate 4. Resin such as epoxy may be used as the material of the substrate 4. The acoustic attenuation member 24 may include resin alone, but may include a resin including fine particles of metal such as titanium or tungsten. In order to prevent reflection, it is preferable to match the acoustic impedance of the substrate 4 to that of the acoustic attenuation member 24.

An operation amplifying circuit and an A/D converter may be mounted on the substrate 4 as is the case in the cell 1. Consequently, the length of wiring before discretization can be reduced, and a reflected wave and noise that are dependent on the length of the wiring can be suppressed.

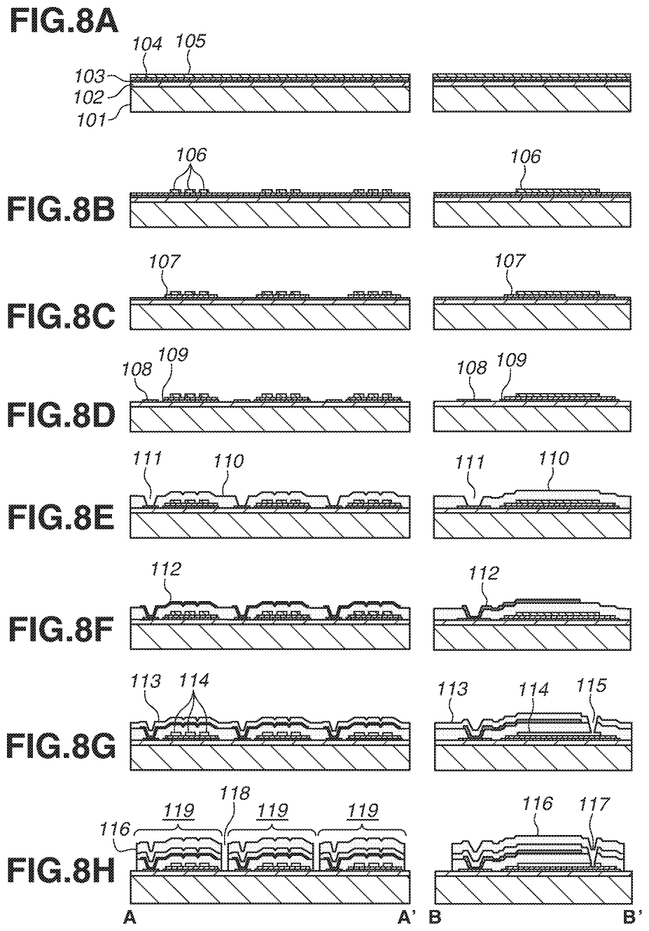

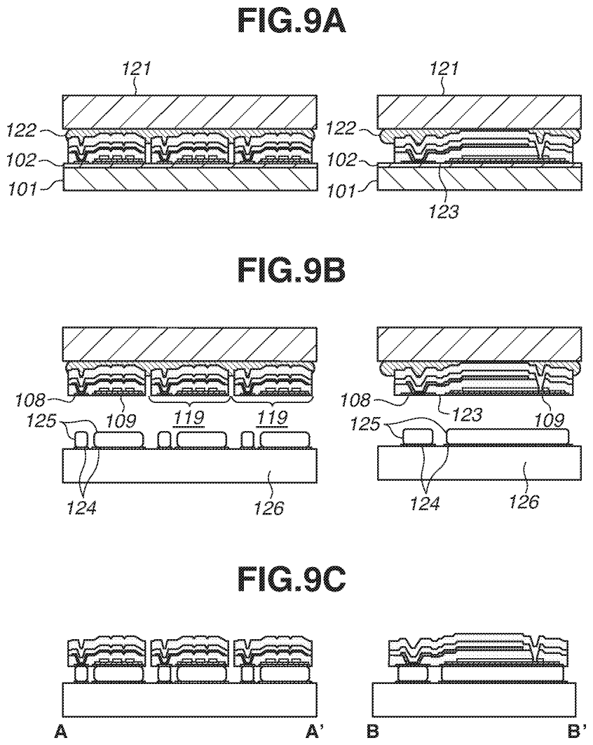

A method of manufacturing a CMUT according to the present exemplary embodiment is described. A manufacturing process of the CMUT according to the present exemplary embodiment includes the steps of manufacturing and separating elements illustrated in FIGS. 8A to 8H, the step of bonding a supporting substrate illustrated in FIG. 9A, the step of removing a wafer illustrated in FIG. 9B, and the steps of bonding a flexible substrate and of removing the supporting substrate illustrated in FIG. 9C.

The step of manufacturing elements is illustrated in FIGS. 8A to 8H. A film 102 is formed on a wafer 101. The film 102 is removed in the step of removing the wafer (FIG. 9B). The wafer 101 may be, for example, monocrystalline silicon, silicon on insulator (SOI), glass, crystallized glass, quartz, or silicon carbide. The film 102 may be an oxide or nitride of the wafer 101. The film 102 may be a deposited film, and may be a metal or insulating film processed through vacuum vapor deposition, sputtering, chemical vapor deposition (CVD), physical vapor deposition, thermal oxidation, or other method. However, in order to remove the film 102 in the following step, the material of the film 102 is preferably a material other than the materials of the element part.

A conductive film 103 to be the first electrode 8 and the electrode 16 in FIGS. 1A to 1C is deposited. The conductive film 103 requires smoothness, conductivity, and heat resistance. The material of the conductive film 103 may be tungsten, molybdenum, titanium, aluminum, neodymium, chromium, cobalt, or a laminate, compound, or alloy of these metals, or a compound or alloy of silicon or copper. Further, a semiconductor or compound semiconductor including a high concentration of impurities is also acceptable. The film deposition method may be vacuum vapor deposition, sputtering, CVD, or other method. A thickness of the conductive film 103 is approximately 50 nanometers to 400 nanometers.

Next, a first insulating film 104 is deposited. The material of the first insulating film 104 may be, for example, silicon oxide or silicon nitride. The film deposition method may be CVD or sputtering. A film 105 to be a sacrificial layer is subsequently deposited. The material of the film 105 may be, for example, chromium, molybdenum, silicon oxide, or amorphous silicon. The thickness of the film 105 is approximately 100 to 500 nanometers. The sacrificial layer 105 is patterned by photolithography. The first insulating film 104 and the conductive film 103 are patterned in a similar fashion (FIGS. 8C and 8D). A second insulating film 110 (second insulating film 11 illustrated in FIG. 1C) is deposited. The second insulating film 110 on an electrode 108 is removed to form a contact portion 111 (FIG. 8E). The second insulating film 110 may be silicon oxide or silicon nitride, and the thickness thereof is 200 to 800 nanometers. A conductive film 112 (second electrode 12 illustrated in FIG. 1C) to be the second electrode 12 is deposited and patterned (FIG. 8F). A desired material of the conductive film 112 is metal. The material may be, for example, tungsten, molybdenum, titanium, aluminum, neodymium, chromium, cobalt, or a compound or alloy of these metals, or a compound or alloy of silicon or copper. The thickness of the conductive film 112 is 100 to 600 nanometers. A third insulating film 113 is deposited. The material of the third insulating film 113 may be silicon oxide or silicon nitride, and the thickness thereof is 200 to 800 nanometers. The third insulating film 113 covers the conductive film 112 completely. An etch-hole 115 is subsequently formed to remove a sacrificial layer 106 (FIG. 8G). A sealing film 116 (sealing film 17 illustrated in FIG. 1C) is deposited. The material of the sealing film 116 is preferably the same as that of the second insulating film 110 or the third insulating film 113 to increase sealing properties. The second insulating film 110, the conductive film 112, the third insulating film 113, and the sealing film 116 on a cavity 114 (cavity 10 illustrated in FIG. 1C) configure a vibrating membrane. The step of removing the sealing film 116 may be included to adjust the thickness of the vibrating membrane. Since the membrane stress as a whole needs to be tensile stress so that the vibrating membrane functions, the second insulating film 110, the conductive film 112, and the third insulating film 113 is formed while stress is controlled. Especially, it is preferable that the membrane stress of the second insulating film 110 and the third insulating film 113 be approximately 0 to 150 megapascals. Consequently, it is possible to prevent the vibrating membrane from being ripped, swelling, or adhering to a bottom surface of the cavity.

The step of separating elements (FIG. 8H) is the step of removing the second insulating film 110, the third insulating film 113, and the sealing film 116 between the elements by etching. At this point, etching process needs to be performed up to the film 102 or the wafer 101. To perform this step smoothly, the materials of the second insulating film 110, the third insulating film 113, and the sealing film 116 are preferably the same, and are desirably different from the material of the film 102.

The step of bonding a supporting substrate illustrated in FIG. 9A is the step of bonding the upper parts of the elements to a supporting substrate 121 with an adhesive 122. An adhesive sheet can be used as the adhesive 122.

The step of removing the wafer (FIG. 9B) is subsequently performed to remove the wafer 101 and the film 102. To this end, a method that removes the wafer 101 and the film 102 from the back surface of the wafer 101 by grinding or etching, or a method that etches the film 102 to peel the wafer 101 off is acceptable. Whichever method is selected, removal by etching with high selectivity is desirable to prevent an element 119 from being subjected to the processing. Therefore, it is desirable that the wafer 101 or the film 102 be different in material from the conductive film 108, a conductive film 109, and the second insulating film 110. The wafer 101 may be monocrystalline silicon, SOI, glass, crystalized glass, quartz, or silicon carbide. The film 102 may be an oxide or nitride of the wafer 101. If the wafer 101 is, for example, a monocrystalline silicon substrate, the film 102 may be silicon oxide. At this point, an exposed portion of the element 119 needs to be formed with a material other than silicon oxide. If the wafer 101 is glass, the film 102 is not required, and thus the wafer 101 can be removed in a similar step. Considering this step, a material of an area 123 where the electrodes on the bottom surface of the element 119 are separated needs to be different from the material of the film 102. If the film 102 is, for example, silicon oxide, silicon nitride may be used for the area 123 where the electrodes are separated.

Next, the step of bonding a flexible substrate is performed. A substrate 126, which has flexibility, is a flexible printed circuit board having wiring, vias, and electrodes. The substrate 126 is electrically and structurally connected to the electrodes on the bottom portion of the element 119. Accordingly, the elements 119 can be arranged highly and densely. The adhesive 122 is preferably a conductive adhesive or metal such as a solder alloy. If the adhesive 122 is a conductive adhesive, the adhesive 122 is applied in advance to the substrate 126 side, and bonding is performed. In a case of, for example, a solder alloy, a pattern is formed in advance on the substrate side. After bonding, reflow soldering is performed.

Lastly, the step of removing the supporting substrate is performed to remove the supporting substrate 121 (FIG. 9C). At this point, the adhesiveness of the adhesive 122 is reduced to peel the supporting substrate 121 off. For example, a heat-foamable resin material is used as the adhesive 122, which facilitates the removal. The substrate 126 may be bent and then bonded and fixed in a curved surface shape as in FIG. 4B, or the substrate 126 may be set to be in a state of being placable on various curved surfaces while maintaining flexibility.

Element parts are formed on a wafer by the method of manufacturing a CMUT of the present exemplary embodiment. Accordingly, a vibrating membrane, an insulating film, and an electrode of good quality can be formed and processed accurately, and a more homogeneous CMUT can be provided. The elements are separated, and transferred to a flexible substrate having electrodes and wiring. Accordingly, the CMUT can also be brought into close contact in various curved surface shapes. The element parts manufactured on the wafer by the micromachining technology are transferred as they are to the flexible substrate. Accordingly, an array of the elements with a small pitch and with accuracy can be manufactured. Since the element pitch is sufficiently small, artifacts occurring due to diffraction can be minimized

The present exemplary embodiment proposes the structure of a CMUT transducer that can make ultrasound diagnoses while being placed on curved surfaces of every type, or that can be mounted on curved surfaces of every type. It has been difficult to place a CMUT in a curved surface shape since the CMUT is mainly fabricated on a wafer. With the structure and manufacturing method of the present exemplary embodiment, a substrate part is removed, and only a thin element part is pasted and electrically connected to a flexible substrate. Accordingly, a CMUT with flexibility and including the elements arranged highly densely can be realized. Thus, an ultrasound probe equipped with such a CMUT can be realized. Consequently, internal information from a region of a living body with a complicated curved surface can be imaged with ultrasound. A living body may include biological regions of structures of human or animals such as abdomen, pelvis, and neck. The internal structures within those regions may include bladder, vessels, liver, spleen, kidneys, pancreas, carotid sheath, and thyroid gland.

More specific exemplary embodiments are given below to describe the disclosure in detail.

An example of a CMUT according to a first exemplary embodiment is described, using FIGS. 1A to 1C. FIG. 1A is a top view of the CMUT according to the present exemplary embodiment. The elements 3 are configured as a two-dimensional array.

Firstly, a cross-sectional structure of the cell 1 is described, using FIG. 1C. A laminated film of tungsten with a thickness of 100 nanometers and titanium with a thickness of 10 nanometers is the first electrode 8. On the laminated film, a silicon oxide as the first insulating layer 9 with a thickness of 400 nanometers is formed. The cavity 10 is 300 nanometers high, and is sealed under a reduced pressure of 200 pascals. The second insulating film 11 on the cavity 10 is a silicon nitride film with a thickness of 600 nanometers. The second electrode 12 includes an alloy of aluminum and neodymium with a thickness of 100 nanometers. The diameter of the vibrating membrane 6 of the cell 1 is 30 micrometers. The diameter of the second electrode 12 is 28 micrometers. The width of the wiring 2 is four micrometers. The third insulating film 13 includes silicon nitride with a thickness of 400 nanometers. The second insulating film 11, the second electrode 12, and the third insulating film 13 are constructional elements of the vibrating membrane 6. The second insulating film 11 and the third insulating film 13 have tensile stress and therefore form the vibrating membrane 6 stably. According to the present exemplary embodiment, the silicon nitride films used as the second insulating film 11 and the third insulating film 13 have a stress of 100 megapascals. The thickness of the sealing film 17 is 700 nanometers. The cavity 10 is sealed in the hole 14. The material of the sealing film 17 is silicon nitride.

The second electrode 12 is extracted in the horizontal direction, and is connected to the electrode 16 on the bottom surface. The electrode 16 is formed of the same material with the same thickness as the first electrode 8, and is insulated by the second insulating film 11 from the electrode 8. The electrodes 8 and 16 are bonded to the flexible substrate 4 from the bottom portion through the conductive adhesive 19. The base material of the substrate 4 is polyimide. The material of the wiring 21 and the via 22 is copper. The conductive adhesive 19 is a mixture of metal fine particles based on epoxy resin. Each element 3 includes at least a pair of the electrodes 8 and 16. The surface electrodes 18 of the substrate 4 face the electrodes 8 and 16. An electrical signal is extracted through the wiring 21 and the via 22 in the substrate 4.

The CMUT 100 applies a DC voltage from the voltage application unit 5 to between the first electrode 8 and the second electrode 12 to perform an electromechanical conversion, and accordingly can transmit and receive ultrasonic waves. When transmitting the ultrasonic waves, the CMUT 100 applies an AC voltage from the voltage application unit 5 in addition to the DC voltage. A driving condition of the CMUT 100 is determined by a pull-in voltage of the cell 1. The “pull-in” indicates that, when a DC voltage is applied to between the first electrode 8 and the second electrode 12, a restoration force based on the rigidity of the vibrating membrane 6 and electrostatic force go out of balance and the vibrating membrane 6 comes into contact with the bottom surface of the cavity 10. This voltage is called a pull-in voltage. The pull-in voltage of the cell 1 according to the present exemplary embodiment is 300 volts. The driving conditions are that the DC voltage is 200 V and the maximum amplitude value of the AC voltage is 100 V.

The element 3 is structurally separated from the other elements 3 by the grooves 20. The width of the groove 20 is five micrometers. The height of the element 3 is approximately 2.3 micrometers right on the cavity 10. Hence, even if the CMUT 100 is bent by 90 degrees at the groove 20 part, the element 3 does not come into contact with the adjacent elements 3. Therefore, the CMUT 100 can be deformed into any type of shape by freely bending the flexible substrate 4.

The main use of a CMUT is to receive an ultrasound echo transmitted by the CMUT itself and acquire various types of information, such as distance, direction, type of an object, characteristics, and velocity. In order to acquire information from various regions of a living body, a transducer is required to be deformable along a complicated curved surface of the living body, and have flexibility that allows the transducer to be mounted on a desired curved surface. In the case of the CMUT 100 of the present exemplary embodiment, an ultrasound probe can be provided in which the elements 3 are placed on a free curved surface with a small pitch.

An example of a CMUT according to a second exemplary embodiment is described with reference to FIGS. 4A and 4B. The CMUT 100 has a configuration where the rectangular elements 3 are arranged one-dimensionally. The length of the short side of the element 3 is 200 micrometers, and the length of the long side thereof is eight millimeters. Cells 1 are close-packed and arranged in the element 3. Each of the elements 3 is structurally separated by grooves 20. The structure of the element 3 in the thickness direction corresponds to the first exemplary embodiment.

The resin 25 is bonded on the top surface side of the element 3 to protect the surface of the element 3. The resin 25 is silicone rubber, and has a thickness of approximately 300 micrometers. The acoustic impedance of the resin 25 is approximately 1.5 Mega Rayls (MRayls), which matches the acoustic impedance of a living body. The resin 28, as an under-fill material, buries between the elements 3 or gaps in a conductive adhesive. The resin 28 increases insulation reliability and structural reliability. The resin 28 is epoxy resin.

The CMUT 100 covered with the resin 25 according to the present exemplary embodiment realizes efficient propagation of ultrasonic waves into and from a living body, and thus a highly reliable ultrasound probe having the elements 3 arranged on a curved surface is obtained.

An example of a CMUT according to a third exemplary embodiment is described with reference to FIG. 5. The CMUT of the present exemplary embodiment corresponds to the one illustrated in FIG. 1.

The elements 3 are bonded and mounted on the substrate 4 having epoxy resin as a base material. The acoustic attenuation member 24 is bonded to the back surface of the substrate 4. The acoustic attenuation member 24 includes epoxy resin where tungsten fine particles are mixed. The acoustic attenuation member 24 absorbs ultrasonic waves produced by the CMUT or entering the CMUT, and thus reflected waves can be reduced. Further, since the substrate 4 itself has acoustic attenuation characteristics according to the present exemplary embodiment, the substrate 4 and the acoustic attenuation member 24 can also be considered to be one unit. Since the reflected waves are noise on an ultrasound image, the CMUT with the acoustic attenuation member 24 of the present exemplary embodiment can provide a high-quality ultrasound image.

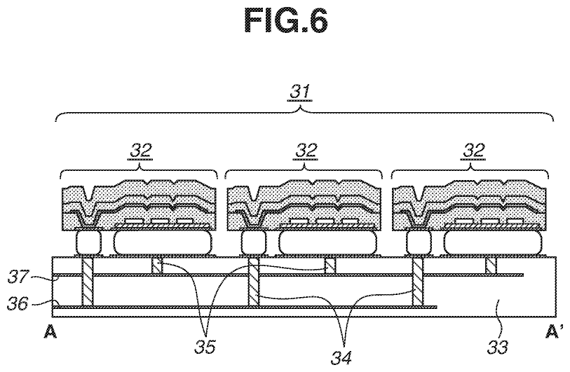

An example of a CMUT according to a fourth exemplary embodiment is described with reference to FIG. 6. The CMUT of the present exemplary embodiment corresponds to the one illustrated in FIG. 1.

Vias 34 and 35 and wirings 36 and 37 are included in a substrate 33, and are electrically connected. An element 31 is structurally divided into sub-elements 32. The sub-elements 32 are electrically connected in parallel in the substrate 33. A first electrode of the sub-element 32 is connected to the wiring 36 through the via 34. A second electrode of the sub-element 32 is connected to the wiring 37 through the via 35. For example, in a case of a linear probe, an acoustic lens focuses in the element 31. However, in a case where high-frequency ultrasonic waves, in which attenuation of the acoustic lens is required to take into consideration, is used, the element 31 is deformed to achieve focusing. In the CMUT according to the present exemplary embodiment, the element 31 can be deformed and mounted. Accordingly, provision of an ultrasound probe that can form an ultrasound beam while the element 31 has a curved surface is realized.

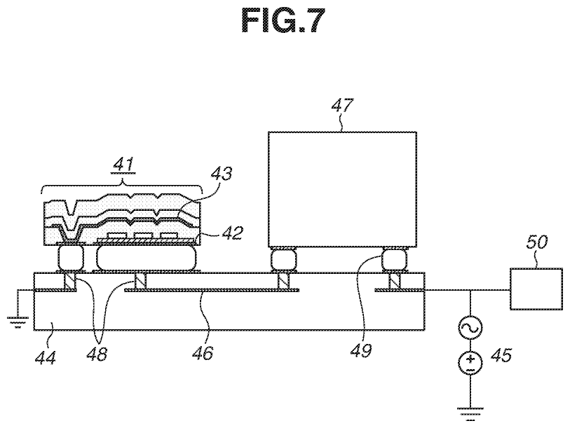

An example of a CMUT according to a fifth exemplary embodiment is described with reference to FIG. 7. The CMUT of the present exemplary embodiment corresponds to the one illustrated in FIG. 1.

A transmission voltage signal is applied by a voltage application unit 45 to a first electrode 42 of an element 41 through a circuit 47 mounted on a substrate 44, wiring 46, a via 48, and a conductive adhesive 49. On the other hand, ultrasonic waves received by the element 41 are converted into an electrical signal. The electrical signal is discretized by an A/D converter in a receiver 50 similarly through the via 48, the wiring 46, and the circuit 47. The circuit 47 includes a changeover switch for transmission and reception. Circuits for transmission and reception are switched automatically. The circuit 47 further includes an operation amplifying circuit that amplifies a received signal.

In the CMUT according to the present exemplary embodiment, the elements 41 can be deformed and mounted. The circuit 47 can also be mounted on the substrate 44. Accordingly, provision of an ultrasound probe that hardly receives influence of noise can be realized.

An example of a method of manufacturing a CMUT according to a sixth exemplary embodiment is described with reference to FIGS. 8A to 8H and FIGS. 9A to 9C. The CMUT manufactured according to the present exemplary embodiment corresponds to the one illustrated in FIG. 1.

In FIG. 8A, the monocrystalline silicon substrate 101 is thermally oxidized to form a silicon oxide film 102 of one micrometer. Although all the substrate surfaces are oxidized in the thermal oxidation step, the back side is not illustrated in FIG. 8A.

Next, sputtering is performed to consecutively deposit tungsten of 100 nanometers in thickness and titanium of 10 nanometers in thickness as a first metal film (i.e., conductive film) 103 (first electrode 8 illustrated in FIG. 1C). A silicon oxide film is subsequently deposited to form 400 nanometers in thickness by plasma-enhanced chemical vapor deposition (PECVD), and thus a first insulating film 104 (first insulating film 9 illustrated in FIG. 1C) is formed. The amorphous silicon film 105 is subsequently deposited.

FIG. 8B illustrates formation of the sacrificial layer 106, which is the step of etching and removing a part of the amorphous silicon film 105. The first insulating film 104 is etched except a part 107 thereof (FIG. 8C). Next, the conductive film 103 is etched to form electrodes 108 and 109 (FIG. 8D). FIG. 8E illustrates a step of forming the second insulating film 110. A silicon nitride film is deposited to 300 nanometers in thickness by PECVD to form a contact hole 111.

FIG. 8F illustrates formation of a second metal film (second electrode) 112. A film of an alloy of aluminum and neodymium is deposited to 100 nanometers in thickness by sputtering to form the second electrode 112.

A silicon nitride film is subsequently deposited to 400 nanometers in thickness by PECVD to form a third insulating film 113 (third insulating film 13 illustrated in FIG. 1C) (FIG. 8G). In order to remove the sacrificial layer 106, a hole 115 that penetrates parts of the second insulating film 110 and the third insulating film 113 is formed in such a manner as to communicate with the sacrificial layer 106. To communicate here is to be in contact, to share or exchange material, or to transfer or exchange constituents of the materials. The amorphous silicon forming the sacrificial layer 106 is removed by dry etching with xenon difluoride to form the cavity 114.

The hole 115 that has been used to etch the sacrificial layer 106 is subsequently sealed by PECVD with a silicon nitride film 116 with a thickness of 700 nanometers (FIG. 8H). At this point, the film deposition condition is an atmosphere of approximately 200 pascals. Thus, the cavity 114 is sealed under a reduced pressure. At the time of sealing, the silicon nitride laminated in the upper part of the vibrating membrane may be removed by etching. If a considerable film thickness is required in the sealing step, an etching stop layer is formed on the upper part of the third insulating film 113 before the etching of the sacrificial layer 106 is performed. After the etching of the sacrificial layer 106, sealing, and the etching of the sealing film 116 are performed, the etching stop layer is removed. The second insulating film 110, the third insulating film 113, and the sealing film 116 between the elements 119 are etched and removed to form a groove 118, and the elements 119 are spatially separated accordingly.

FIG. 9A illustrates a step of bonding a supporting substrate. Top surfaces of the separated elements 119 are pasted to the supporting substrate 121 with a heat-foamable adhesive 122. FIG. 9B illustrates the step of removing a wafer. The silicon oxide film 102 is etched with hydrofluoric acid to remove a wafer 101. Next, the step of bonding the flexible substrate is illustrated. A conductive adhesive 125 is applied by screen printing to a flexible substrate 126 having electrodes 124 on a surface thereof. The conductive adhesive 125 and electrodes 108 and 109 are subsequently bonded together. Lastly, the heat-foamable adhesive 122 is heated and foamed to remove the supporting substrate 121. Consequently, the CMUT where the elements 119 are highly densely mounted on the flexible substrate 126 is completed (FIG. 9C).

The main use of a CMUT is to receive an ultrasound echo transmitted by the CMUT itself and acquire various types of information, such as distance, a direction, type of an object, characteristics, and a velocity. In order to acquire information from various regions of a living body, a transducer is required to be deformable along a complicated curved surface of the living body, and have flexibility that allows the transducer to be mounted on a desired curved surface. The method of manufacturing a CMUT according to the present exemplary embodiment allows providing of an ultrasound probe where the elements 119 are placed on a free curved surface with a small pitch.

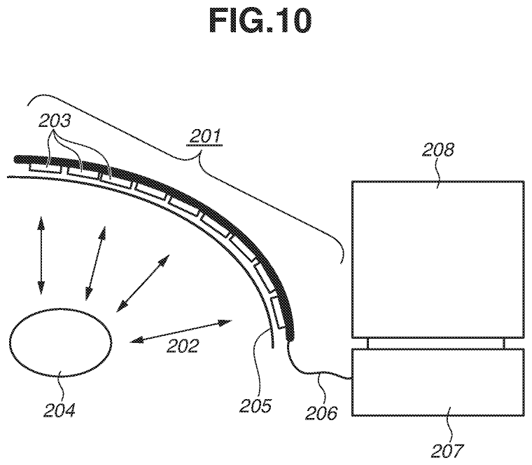

An example of an ultrasound image forming apparatus according to a seventh exemplary embodiment is described with reference to FIG. 10. The configurations of a CMUT 201 and an element 203, which are used in the present exemplary embodiment, are similar to those of the first to sixth exemplary embodiments.

A CMUT 201 is deformed and installed in such a manner as to fit a living body surface 205 having a curved surface. Gel whose acoustic impedance is close to that of the living body is placed between the living body surface 205 and an element 203, thereby ultrasonic waves 202 are propagated into or from the living body. The ultrasonic waves 202 produced by the element 203 are returned to the element 203 again as an echo from an acoustic reverberation body 204 in the living body, and are received. The received ultrasonic waves are converted by the element 203 into an electrical signal. The electrical signal is transferred to a processing unit (e.g., calculator) 207 via wiring 206 connected to the outside through the wiring in the substrate. The processing unit 207 converts the obtained signal into image information. A display unit (e.g., display) 208 displays an ultrasound image in real time. A display control unit (not illustrated) controls display of an image on the basis of the image information obtained by the display unit 208 and the processing unit 207.

As described above, the ultrasound image forming apparatus according to the present exemplary embodiment allows providing of an image forming apparatus that can form a high-quality image with the CMUT 201 that can adjust a shape thereof to a complicated surface shape of a living body.

Since the CMUT according to the exemplary embodiments is installed on the flexible substrate, the CMUT can be bent and placed on a region of a living body. Moreover, the wiring installed on the flexible substrate connects the elements electrically. Accordingly, it is possible to have a small pitch between the elements, and thus an ultrasound signal can be acquired which can form a high-quality ultrasound image where artifacts caused by the diffraction of ultrasonic waves are suppressed.

While the disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2018-185433, filed Sep. 28, 2018, which is hereby incorporated by reference herein in its entirety.

What is claimed is:

1. A capacitive transducer comprising at least one element,
wherein the element includes at least one cell,
wherein the at least one cell includes a first electrode, a first insulating film on the first electrode, and a vibrating membrane having a second insulating film and a second electrode, which face the first insulating film across a cavity,
wherein the element is provided on a flexible substrate,
wherein the substrate includes, on a surface thereof, a surface electrode and wiring electrically connected to the surface electrode, and
wherein the first and second electrodes are electrically connected to the surface electrode.

2. The capacitive transducer according to claim 1, wherein at least one of the first and second electrodes is electrically connected to the surface electrode by point-to-point construction.

3. The capacitive transducer according to claim 1, wherein at least one of the first and second electrodes is electrically connected to the surface electrode by pattern wiring.

4. The capacitive transducer according to claim 1,
wherein the first and second electrodes are electrically extracted to reach the substrate side of the element, and
wherein the first and second electrodes are connected to the surface electrode via a conductive adhesion layer.

5. The capacitive transducer according to claim 4, wherein the adhesion layer includes conductive resin.

6. The capacitive transducer according to claim 4, wherein the adhesion layer includes an alloy.

7. The capacitive transducer according to claim 4, wherein the first and second electrodes are insulated by a part of the second insulating film on a surface on the substrate side of the element.

8. The capacitive transducer according to claim 4,
wherein the first insulating film includes silicon oxide, and
wherein the second insulating film includes silicon nitride.

9. The capacitive transducer according to claim 1,
wherein the capacitive transducer comprises a plurality of the elements, and
wherein a height of the element from a surface on the substrate side to a surface opposite to the substrate side is less than a distance between adjacent elements of the plurality of the elements.

10. The capacitive transducer according to claim 1,
wherein a height of the element from a surface on the substrate side to a surface opposite to the substrate side is three micrometers or less, and
wherein a distance between adjacent elements of a plurality of the elements is twice or less the height.

11. The capacitive transducer according to claim 1,
wherein the capacitive transducer comprises a plurality of the elements, and
wherein the wiring on the substrate connects the first electrodes or the second electrodes of the plurality of the elements to electrically connect the plurality of the elements, and
wherein the substrate has curvature.

12. The capacitive transducer according to claim 1, wherein the substrate includes epoxy resin.

13. The capacitive transducer according to claim 1, wherein an acoustic attenuation member is bonded to a back surface of the substrate.

14. The capacitive transducer according to claim 13, wherein the acoustic attenuation member includes epoxy resin in which fine particles of tungsten are mixed.

15. The capacitive transducer according to claim 1, wherein the vibrating membrane is covered on a top surface thereof with resin.

16. The capacitive transducer according to claim 1, wherein an operation amplifier configured to amplify an electrical signal transmitted from the first or second electrode is connected to a part of the substrate.

17. An image forming apparatus comprising:
the capacitive transducer according to claim 1;
a processing unit configured to perform a process of generating image information from an electrical signal received by the element; and
a display control unit configured to display the image information on a display unit,
wherein the capacitive transducer is provided in a curved surface shape with flexibility of the substrate.

18. A method of manufacturing a capacitive transducer including a cell having a first electrode, a first insulating film on the first electrode, and a vibrating membrane having a second insulating film and a second electrode, which face the first insulating film across a cavity, and an element, in which a plurality of cells is electrically connected, the method of manufacturing the capacitive transducer comprising:
forming a plurality of the elements on a wafer;
spatially separating the plurality of the elements that are adjacently positioned;
bonding the wafer and a supporting substrate on a side on which the elements are formed;
removing the wafer;
bonding the elements to a flexible substrate; and
removing the supporting substrate from the elements.

19. The method of manufacturing the capacitive transducer according to claim 18, wherein the forming the plurality of the elements on the wafer includes
the wafer being monocrystalline silicon,
depositing a first silicon oxide film,
depositing a first metal film,
depositing a second silicon oxide film,
depositing and forming a sacrificial layer,
etching the second silicon oxide film,
etching the first metal film to form a bottom portion of the first electrode and a bottom portion of the second electrode,
depositing a first silicon nitride film,
depositing a second metal film and forming the second electrode,
depositing a second silicon nitride film,
forming a hole penetrating the first and second silicon nitride films in such a manner as to communicate with the sacrificial layer,
removing the sacrificial layer through the hole to form the cavity, and
depositing a third silicon nitride film to seal the hole.

20. The method of manufacturing the capacitive transducer according to claim 18, wherein the spatially separating the plurality of the adjacent elements includes etching a material forming the second insulating film.

21. The method of manufacturing the capacitive transducer according to claim 18, wherein the removing the wafer includes etching the first silicon oxide film.

22. The method of manufacturing the capacitive transducer according to claim 18,
wherein the bonding the wafer and the supporting substrate includes bonding the wafer and the supporting substrate with an adhesive sheet, and
wherein the removing the supporting substrate from the elements includes removing the elements from the supporting substrate by application of heat.

23. The method of manufacturing the capacitive transducer according to claim 18,
wherein the bonding the elements to the flexible substrate includes connecting each of the first electrode on a bottom portion of the element and the second electrode to an electrode on a surface of the substrate, using a conductive adhesive, and
wherein the first and second electrodes are electrically separated.

24. The method of manufacturing the capacitive transducer according to claim 18,
wherein the bonding the elements to the flexible substrate includes connecting each of the first electrode on a bottom portion of the element and the second electrode to an electrode on a surface of the substrate, using a solder alloy, and
wherein the first and the second electrodes are electrically separated.
